(19) United States
(12) Patent Application Publication
(10) Pub. No.: US 2007/0289595 A1
(43) Pub. Date: Dec. 20, 2007

(54) Breathing apparatus for person with stoma

(76) Inventor: John Lubelski, Toronto (CA)

Correspondence Address: BERESKIN AND PARR, 40 KING STREET WEST, BOX 401, TORONTO, ON, M5H 3Y2, US

(21) Appl. No.: 11/450,375

(22) Filed: Jun. 12, 2006

(51) Int. Cl. A61M 16/00

(52) U.S. Cl. 128/207.14; 128/200.26

(57) ABSTRACT

An apparatus for a person with a stoma is disclosed. The apparatus includes a substantially waterproof garment having a sealed area defined by a boundary, such that the stoma is located within the boundary. The boundary is in sealed engagement with the person. The sealed area of the waterproof garment defines an opening near the stoma. The apparatus also includes an air tube received in the opening. The air tube includes an interior portion located inwardly of the opening and an exterior portion located outwardly of the opening. The interior portion of the air tube is received in the stoma. A flange located on the interior portion. A nut threadably engages a threaded portion of the exterior portion to urge the flange against the waterproof garment surrounding the opening, thereby forming a seal between the air tube and the opening in the waterproof garment. A flexible air hose is also provided. The first end of the air hose is in sealed engagement with the exterior portion of the air tube and in fluid communication therewith.

### FIELD OF THE INVENTION

The present invention relates to a breathing apparatus for a person with a tracheotomy stoma. In particular, the present invention relates to a breathing apparatus to enable a person with a tracheotomy stoma to participate in swimming activities, water sports, or the like.

### BACKGROUND OF THE INVENTION

It can be unsafe for a person who has had a tracheotomy to participate in swimming or other water sport activities. This is due to the risk of water entering the person's lungs through the stoma. The stoma is a surgically created opening in the person's neck to permit air to pass directly through the trachea.

Several devices to assist a person with a stoma to swim are known. The prior art devices have the disadvantage of requiring a watertight seal between the stoma tube and the stoma. Such a seal is difficult to establish and maintain, particularly during periods of high activity by the user.

Accordingly, there is a need for an improved breathing apparatus to enable a person with a tracheotomy stoma to participate in swimming, water sports, or other water-related activities.

### SUMMARY OF THE INVENTION

According to a first aspect of the invention, an apparatus for a person with a stoma is provided. The apparatus comprises:

a) a substantially waterproof material comprising a sealed area defined by a boundary, wherein the stoma is located within the boundary, the boundary being in sealed engagement with the person, the sealed area of the waterproof material defining an opening proximate to the stoma;

b) an air conduit received in the opening, the air conduit comprising an interior portion located inwardly of the opening and an exterior portion located outwardly of the opening, the interior portion being adapted for fluid communication with the stoma, wherein the air conduit is in sealed engagement with the opening in the waterproof material; and

c) an air line defining a first end and a second opposing end, wherein the first end is in sealed engagement with the exterior portion of the air conduit and in fluid communication therewith.

Preferably, the air conduit is in unsealed fluid communication with the stoma.

In one embodiment, the apparatus further comprises a mouthpiece in sealed engagement with the second end of the air line and in fluid communication therewith. Preferably, the apparatus also comprises a valve having first and second valve ends. The first valve end is in sealed engagement and in fluid communication with the air line. The second valve end is in sealed engagement and in fluid communication with the mouthpiece. The valve is preferably operable between a sealed position and an open position where air is permitted to escape from the valve to atmosphere, wherein the valve is in the sealed position when the person inhales and in the open position when the person exhales.

In another embodiment, the apparatus further comprises a snorkel tube which is sealingly connected to the second end of the air line or to the second valve end.

According to a second aspect of the invention, an apparatus for a person with a stoma is provided. The apparatus comprises:

a) a substantially waterproof garment comprising a sealed area defined by a boundary, wherein the stoma is located within the boundary, the boundary being in sealed engagement with the person, the sealed area of the waterproof garment defining an opening proximate to the stoma;

b) an air tube received in the opening, the air tube comprising:
i) an interior portion located inwardly of the opening, the interior portion being adapted for unsealed fluid communication with the stoma;
ii) an exterior portion located outwardly of the opening;
iii) a flange located on the interior portion; and
iv) a nut, wherein the nut threadably engages a threaded portion of the exterior portion to urge the flange against the waterproof garment surrounding the opening, thereby forming a seal between the air tube and the opening in the waterproof garment; and
   a flexible air hose defining a first end and a second opposing end, wherein the first end is in sealed engagement with the exterior portion of the air tube and in fluid communication therewith.

Preferably, the air tube is in unsealed fluid communication with the stoma.

In one embodiment, the apparatus further comprises a mouthpiece in sealed engagement with the second end of the air hose and in fluid communication therewith. Preferably, the apparatus also comprises a valve having first and second valve ends. The first valve end is in sealed engagement and in fluid communication with the air hose. The second valve end is in sealed engagement and in fluid communication with the mouthpiece. The valve is preferably operable between a sealed position and an open position where air is permitted to escape from the valve to atmosphere, wherein the valve is in the sealed position when the person inhales and in the open position when the person exhales.

### BRIEF DESCRIPTION OF THE DRAWINGS

In the accompanying drawings:

FIG. 1 is a perspective view of an apparatus according to a preferred embodiment of the present invention;

FIG. 2 is an exploded perspective view of the preferred embodiment;

FIG. 3 is a plan view of a valve for the preferred embodiment;

FIG. 4 is a cross-sectional view of the valve of FIG. 3 along line 4-4; and

FIG. 5 is a perspective view of an alternative embodiment of the present invention.

### DETAILED DESCRIPTION OF THE INVENTION

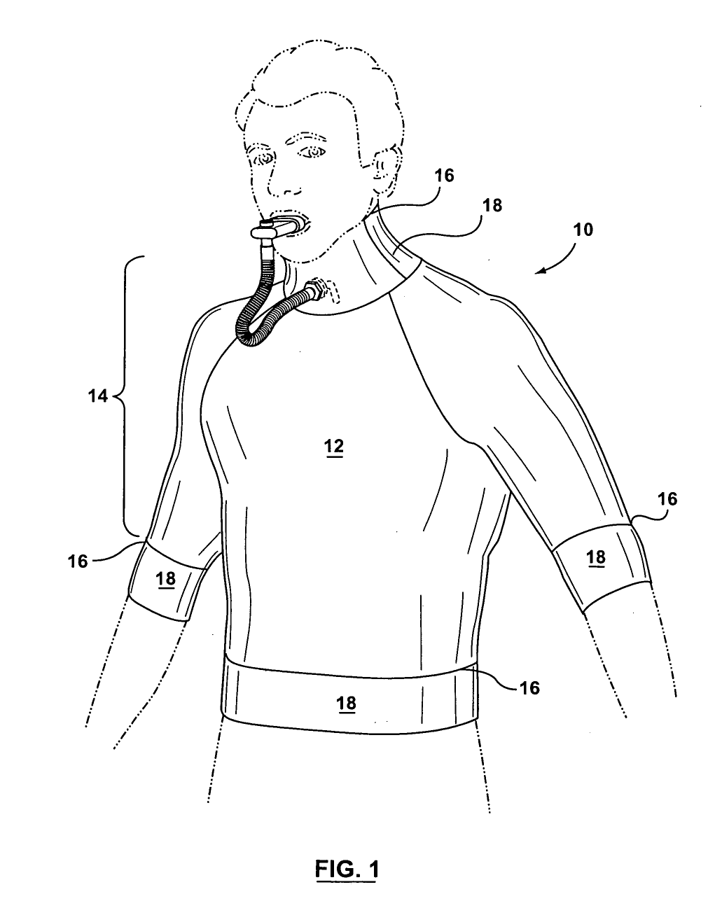

FIG. 1 shows a breathing apparatus 10 to enable a person with a stoma to participate in swimming, water sports, and other water-related activities. A tracheotomy stoma is a surgical opening in the neck. A person who has had this type of tracheotomy procedure breathes through the stoma.

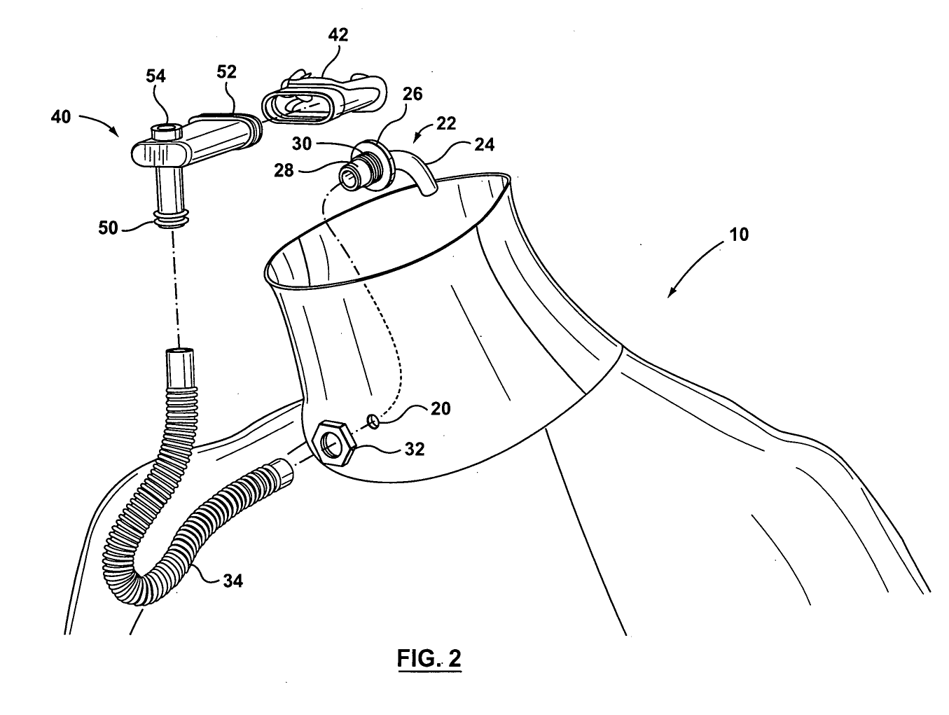

Referring now to FIGS. 1 and 2, the apparatus 10 includes a waterproof material, such as a waterproof garment 12. The waterproof garment 12 includes a sealed area 14 within a boundary 16. The boundary 16 forms a waterproof seal with the person's body. In a preferred embodiment, the boundary 16 is formed by foldable rubber cuffs 18 located at the torso, neck, and arms of the garment 12, respectively. The sealed area 14 is formed by the portion of the garment sealed by the cuffs 18. In an exemplary embodiment, the garment 12 is a drysuit made from 3 mm or 5 mm neoprene rubber. Preferably, the drysuit includes a waterproof zipper (not shown) along the back to assist with putting on the drysuit and taking it off. It will be understood by those skilled in the art that the waterproof material may be any other suitable garment, or may not be a garment at all, but simply a piece of material sealed against an area of the person's body surrounding the stoma.

The garment 12 includes an opening 20 adjacent to the stoma. An air conduit, such as an air tube 22, is received in the opening 20. An interior portion 24 of the air tube 22 includes a flange 26 at one end which locates near the opening 20. The remainder of the interior portion 24 of the air tube 22 is received in the stoma. Preferably, the interior portion has a diameter of 0.9 mm to 1.6 mm to accommodate most stoma sizes, including children.

An exterior portion 28 of the air tube 22 projects out of the opening 20 in the garment 12. The exterior portion 28 includes a threaded portion 30 adjacent to the opening 20. A threaded nut 32 is screwed onto the threaded portion 28 to squeeze the portion of the garment 12 surrounding the opening 20 against the flange 26 to create a watertight seal.

The air tube 22 is in fluid communication with the stoma, such that air being inhaled and exhaled passes through the air tube 22. For the reasons described below, a watertight seal between the air tube 22 and stoma is not required.

An air line, such as a flexible air hose 34, is connected to a distal end of the exterior portion 28 of the air tube 22. Preferably, the air hose 34 is a conventional corrugated, clear medical grade extruded plastic hose having an internal diameter of 15 mm and a length of 12 inches. The air hose 34 is finished at both ends so as to provide a sealed fit with the exterior portion 28 of air tube 22 and a first valve end 50 (described below). The clear color enables the user to examine the interior of the tube for unwanted material, such as mucus.

Preferably, the other end of the air hose 34 is connected to a valve 40. The valve 40 is in turn connected to any suitable mouthpiece 42, such as a commercially available mouthpiece available from Seabreeze™, Innovative Scuba™, TUSA™, or Seacure™. A sealed connection between the mouthpiece 42, valve 40, air hose 34, and air tube 22 is provided in any suitable fashion.

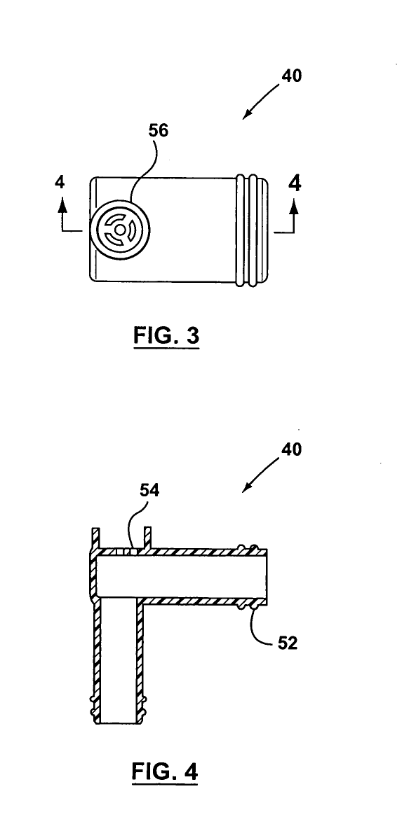

Referring now to FIGS. 2-4, the valve 40 includes a first valve end 50 in sealed engagement with the air hose 34 and a second valve end 52 in sealed engagement with the mouthpiece 42. In a preferred embodiment, the first valve end 50 has an inner diameter of 11 mm and an outer diameter of 15 mm to connect to the air hose 34. The second valve end 52 has a an oval shape having a height of 17 mm and a width of 33 mm to mate with a commercially available mouthpiece 42. The valve 40 also includes a valve outlet 54 covered by a conventional rubber flap 56. It will be understood by those skilled in the art that the valve may be of any other type valve, such as other types of one way valves.

The operation of the preferred embodiment of the present invention will now be described with reference to FIGS. 1-4.

The person breathes through his or her nose. Upon inhaling, the air enters through the nose and flows out of the mouth, into the mouthpiece 42, through the valve 40, air hose 34, and air tube 22 into the stoma. Upon exhaling, the air traveling out through the air hose can be expelled to the atmosphere through the valve outlet 54. This makes breathing using the apparatus 10 more convenient for the person.

Upon inhaling, the pressure in the valve 40 is below atmospheric pressure. Accordingly, the flap 56 covering the valve outlet 54 remains in the sealed position. Upon exhaling, the pressure in the valve 40 is above atmospheric. The pressure urges the flap 56 away from the valve outlet 54 to open the valve 40 and permit air to escape.

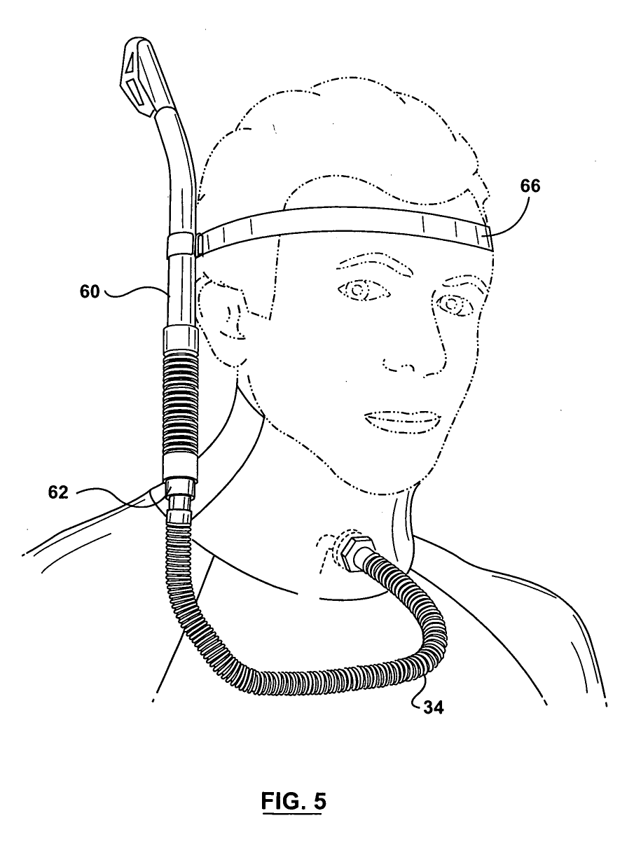

FIG. 5 shows another alternative embodiment of the invention particularly suitable for snorkeling. Like parts are assigned like reference numbers. In this embodiment, a mouthpiece is not necessary. Instead, a conventional snorkel tube 60 is connected to the air hose 34 by any suitable reducer 62. Any suitable retainer, such as a headband 66, may be provided to maintain the snorkel tube 60 in a generally vertical position.

The present invention provides the advantage of eliminating the need for maintaining a seal between the stoma tube and the stoma. Such a seal is difficult to maintain during vigorous swimming or other water sports activities.

While the present invention as herein shown and described in detail is fully capable of attaining the above-described objects of the invention, it is to be understood that it is the presently preferred embodiment of the present invention and thus, is representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it is to be encompassed by the present claims.

1. An apparatus for a person with a stoma, the apparatus comprising:
a) a substantially waterproof material comprising a sealed area defined by a boundary, wherein the stoma is located within the boundary, the boundary being in sealed engagement with the person, the sealed area of the waterproof material defining an opening proximate to the stoma;
b) an air conduit received in the opening, the air conduit comprising an interior portion located inwardly of the opening and an exterior portion located outwardly of the opening, the interior portion being adapted for fluid communication with the stoma, wherein the air conduit is in sealed engagement with the opening in the waterproof material; and
c) an air line defining a first end and a second opposing end, wherein the first end is in sealed engagement with the exterior portion of the air conduit and in fluid communication therewith.

2. The apparatus of claim 1, wherein the air conduit is in unsealed fluid communication with the stoma.

3. The apparatus of claim 1, wherein the waterproof material comprises a garment worn by the person.

4. The apparatus of claim 3, wherein the garment is sealed against the person along a neck, torso, and arms of the person.

5. The apparatus of claim 4, wherein the garment is made from neoprene.

6. The apparatus of claim 5, wherein the neoprene has a thickness of about 3 mm.

7. The apparatus of claim 5, wherein the neoprene has a thickness of about 5 mm.

8. The apparatus of claim 4, wherein the garment comprises a plurality of rubber cuffs located along at the neck, waste, and arms, wherein the plurality of rubber cuffs are adapted to seal against the user upon folding of the cuffs.

9. The apparatus of claim 3, wherein the garment is a drysuit.

10. The apparatus of claim 2, further comprising a mouthpiece in sealed engagement with the second end of the air line and in fluid engagement therewith.

11. The apparatus of claim 10, wherein the air conduit comprises an air tube.

12. The apparatus of claim 11, further comprising a nut and a flange located on the interior portion of the air tube, wherein the nut threadably engages a threaded portion of the exterior portion of the air tube to urge the flange against the waterproof material surrounding the opening, thereby forming a seal between the air tube and the opening in the waterproof material.

13. The apparatus of claim 12, wherein the air line comprises a flexible air hose.

14. The apparatus of claim 10, further comprising a valve, the valve comprising a first valve end and a second valve end.

15. The apparatus of claim 14, wherein the first valve end is in sealed engagement and in fluid communication with the air line, wherein the second valve end is in sealed engagement and in fluid communication with the mouthpiece, wherein the valve is operable between a sealed position and an open position where air is permitted to escape from the valve to atmosphere, wherein the valve is in the sealed position when the person inhales and in the open position when the person exhales.

16. The apparatus of claim 15, wherein the valve is in the sealed position when the pressure in the air line is below atmospheric pressure, and in the open position when the air in the valve is above atmospheric pressure.

17. The apparatus of claim 15, wherein the valve comprises a one-way flap valve.

18. The apparatus of claim 2, further comprising a snorkel tube sealingly connected to the second end of the air line and in fluid communication therewith.

19. The apparatus of claim 18, further comprising a retainer adapted to maintain the snorkel tube in a generally vertical position.

20. The apparatus of claim 14, further comprising a snorkel tube sealingly connected to the second valve end and in fluid communication therewith.

21. The apparatus of claim 20, further comprising a retainer adapted to maintain the snorkel tube in a generally vertical position.

22. An apparatus for a person with a stoma, the apparatus comprising:
a) a substantially waterproof garment comprising a sealed area defined by a boundary, wherein the stoma is located within the boundary, the boundary being in sealed engagement with the person, the sealed area of the waterproof garment defining an opening proximate to the stoma;
b) an air tube received in the opening, the air tube comprising:
i) an interior portion located inwardly of the opening, the interior portion being adapted for unsealed fluid communication with the stoma;
ii) an exterior portion located outwardly of the opening;
iii) a flange located on the interior portion; and
iv) a nut, wherein the nut threadably engages a threaded portion of the exterior portion to urge the flange against the waterproof garment surrounding the opening, thereby forming a seal between the air tube and the opening in the waterproof garment; and
c) a flexible air hose defining a first end and a second opposing end, wherein the first end is in sealed engagement with the exterior portion of the air tube and in fluid communication therewith.

23. The apparatus of claim 22, further comprising a mouthpiece in sealed engagement with the second end of the air line and in fluid engagement therewith.

24. The apparatus of claim 22, wherein the garment is sealed against the person along a neck, torso, and arms of the person.

25. The apparatus of claim 23, wherein the garment is made from neoprene.

26. The apparatus of claim 24, wherein the neoprene has a thickness of about 3 mm.

27. The apparatus of claim 24, wherein the neoprene has a thickness of about 5 mm.

28. The apparatus of claim 24, wherein the garment comprises rubber cuffs along at the neck, torso, and arms, the rubber cuffs are adapted to seal against the user upon rolling of the cuffs.

29. The apparatus of claim 22, wherein the garment is a drysuit.

30. The apparatus of claim 21, further comprising a valve, the valve comprising a first valve end and a second valve end.

31. The apparatus of claim 30, wherein the first valve end is in sealed engagement and in fluid communication with the air hose, wherein the second valve end is in sealed engagement and in fluid communication with the mouthpiece, wherein the valve is operable between a sealed position and an open position where air is permitted to escape from the valve to atmosphere, wherein the valve is in the sealed position when the person inhales and in the open position when the user exhales.

32. The apparatus of claim 31, wherein the valve is in the sealed position when the pressure in the air line is below atmospheric pressure, and in the open position when the air in the valve is above atmospheric pressure.

33. The apparatus of claim 32, wherein the valve comprises a one-way flap valve.
